(19) United States
(12) Patent Application Publication
(10) Pub. No.: US 2020/0353006 A1
(43) Pub. Date: Nov. 12, 2020

(54) PHARMACEUTICAL COMPOSITION FOR TREATING CARDIAC HYPERTROPHY

(71) Applicant: Gwo Xi Stem Cell Applied Technology Co., Ltd., Zhubei (TW)

(72) Inventors: Tsung-Ming Lee, Zhubei (TW); Ming-Hsi Chuang, Zhubei (TW); Chun-Hung Chen, Zhubei (TW); Po-Cheng Lin, Zhubei (TW); Chia-Hsin Lee, Zhubei (TW)

(21) Appl. No.: 16/503,614

(22) Filed: Jul. 4, 2019

(30) Foreign Application Priority Data
May 8, 2019 (TW) 108115906

(51) Int. Cl. A61K 35/28; A61K 9/00; A61P 9/12; C12N 5/0775

(52) U.S. Cl. CPC A61K 35/28; A61K 9/0019; C12N 2501/999; C12N 5/0667; A61P 9/12

(57) ABSTRACT

The present invention relates to a pharmaceutical composition for treating cardiac hypertrophy, comprising at least a stem cell and a pharmaceutically acceptable vehicle, wherein the stem cell is prepared by a pretreatment reaction of reacting with an n-butylidenephthalide (BP). The pharmaceutical composition of the present invention can be administered into a body of a hypertensive patient by remote intramuscular injection, so as to reduce superoxide content in the myocardium, increase STAT3 activity, and increase the content of M2 macrophages that promote inflammation resolution, and further effectively treat the symptoms of cardiac hypertrophy caused by hypertension.

### CROSS-REFERENCE TO RELATED APPLICATIONS

The present application claims priority to Taiwanese Patent Application No. 108115906 filed on May 8, 2019, the entire contents of which are hereby incorporated by reference.

### REFERENCE TO SEQUENCE LISTING

A Sequence Listing is submitted as an ASCII formatted text file via EFS-Web, with a file name of “Sequence_listing.TXT”, a creation date of Jul. 4, 2019, and a size of 3,378 bytes. The Sequence Listing filed via EFS-Web is part of the specification and is incorporated in its entirety by reference herein.

### STATEMENT REGARDING PRIOR DISCLOSURES BY AN INVENTOR OR JOINT INVENTOR

This invention was described in a printed publication by inventor on Aug. 28, 2018 entitled “Remote transplantation of human adipose-derived stem cells induces regression of cardiac hypertrophy by regulating the macrophage polarization in spontaneously hypertensive rats” in European Heart Journal, Volume 39, Issue suppl_1, August 2018, ehy563.P4756 and on Mar. 21, 2019 entitled “Remote transplantation of human adipose-derived stem cells induces regression of cardiac hypertrophy by regulating the macrophage polarization in spontaneously hypertensive rats” in Redox Biology, https://doi.org/10.1016/j.redox.2019.101170.

### BACKGROUND OF THE INVENTION

### 1. Field of the Invention

The present invention relates to a pharmaceutical composition, in particular, to a pharmaceutical composition for treating cardiac hypertrophy.

### 2. Description of the Related Art

Left ventricular hypertrophy (LVH) is the most common outstanding damage caused by hypertension in the heart. Left ventricular hypertrophy secondary to essential hypertension is pathologically caused by stimulated growth of cardiomyocytes and activation of fibroblasts leading to an interstitial fibrosis. Clinically, cardiac hypertrophy may further increase the risk of stroke, chronic renal failure, ventricular dysfunction, and sudden death etc.

Spontaneously Hypertensive Rat (SHR) is the most widely used animal model of human essential hypertension. Studies have confirmed that reactive oxygen species (ROS), including superoxide anion, have been proofed to play an early role in ventricular hypertrophy during the pre-SHR hypertrophy phase, and mitochondrial dysfunction is an important cause to generate ROS and a common change in hypertension. Systolic blood pressure at 40 days of SHR shows a significant increase compared with age-matched normotensive rats, and are evaluated to confirm that the significant symptoms of left ventricular hypertrophy can be confirmed by the morphology and evaluating the molecular markers of ventricular hypertrophy at 2 months. In view of the fact that left ventricular hypertrophy is a powerful predictor of cardiovascular disease morbidity and mortality, the symptoms improvement of left ventricular hypertrophy is generally considered to be a target for anti-hypertensive treatment.

Currently, the most important clinical treatments for ventricular hypertrophy and fibrosis include the use of β blockers and angiotensin converting enzyme inhibitors (ACEI).

β blockers can antagonize sympathetic nerve activity, lower heart rates, and reduce myocardial oxygen consumption, so hypertensive patients can significantly improve cardiac ejection fraction to reverse ventricular hypertrophy if taking it for a long time. However, if the drug is suddenly discontinued after long-term use of β receptor blockers, the original symptoms will be often worsened, leading to elevated blood pressure, rapid arrhythmia, increased angina, and even myocardial infarction. In addition, for diabetic patients who are using insulin therapy, the use of β receptor blockers delays the rate of glycemic recovery after insulin-induced hypoglycemia, which is a hypoglycaemic reaction, so patients with diabetes or hypoglycemia should use these drugs with caution.

ACEI-type drugs mainly inhibit the activity of angiotensin-converting enzyme (ACE), reduce the generation of angiotensin (Ang-II), and reduce the hydrolysis of bradykinin (Bradykinin), causing vasodilation, decreased blood volume, decreased blood pressure, and have been clearly demonstrated to reduce left ventricular hypertrophy. However, with the increase of the concentration of bradykinin, the patient may develop severe irritating dry cough, which causes discomfort. In addition, side effects such as taste abnormalities, neutropenia, rash, fever, and angioedema may occur after the administration of ACEI.

Therefore, there is an urgent need to develop a composition that can solve the defects of the current cardiac hypertrophy treatment technology and can fully exert excellent medical effects, in particular, a therapeutic agent for cardiac hypertrophy having a combination of different mechanisms of action.

### BRIEF SUMMARY OF THE INVENTION

In view of this, through various research on various possible solutions for solving traditional technical problems, the inventors further develop a pharmaceutical composition for treating cardiac hypertrophy, which may not only improve the problem of the above conventional technology, but also be used for the deficiencies of the prior art. With the stem cell prepared by pretreatment reaction of n-butylidenephthalide, and remote intramuscular injection (limbs and hips), the pharmaceutical composition of the present invention may effectively reduce superoxide content in the myocardium, increase STAT3 activity, and increase the content of M2 macrophages that promote inflammation regression, and further reverses cardiac hypertrophy, and thus the present invention has been completed.

In other words, the present invention relates to a pharmaceutical composition for treating cardiac hypertrophy, comprising at least a stem cell and a pharmaceutically acceptable vehicle, wherein the stem cell is prepared by a pretreatment reaction of reacting with an n-butylidenephthalide (BP).

According to an embodiment of the present invention, the concentration of n-butylidenephthalide in the pretreatment reaction is in the range of 7 μg/mL to 40 μg/mL.

According to an embodiment of the present invention, the condition of the pretreatment reaction comprises reacting the stem cell with the n-butylidenephthalide at a temperature range of 20 to 40° C., and in addition, the reaction time is in the range of 6 hours to 24 hours.

According to an embodiment of the present invention, the stem cell is at least one selected from a group consisting an umbilical cord blood stem cell, a peripheral blood stem cell, a neural stem cell, an adipose stem cell, and a bone marrow stem cell.

Furthermore, the present invention may provide a method of using a stem cell in preparation of a pharmaceutical composition for treating cardiac hypertrophy, comprising administering an effective amount of a stem cell into a desired individual, wherein the stem cell is prepared by a pretreatment reaction of n-butylidenephthalide.

According to an embodiment of the present invention, the effective dose is in the range of 1×10⁶˜1×10⁸ stem cells, preferably is in the range of 1×10⁶˜1×10⁷ stem cells.

According to an embodiment of the present invention, the cardiac hypertrophy is caused by spontaneous hypertension.

According to an embodiment of the present invention, the stem cell is administered via remote intramuscular injection.

According to an embodiment of the present invention, the desired individual is human or mammal.

### BRIEF DESCRIPTION OF THE SEVERAL VIEWS OF DRAWING

FIGS. 1A to 1F are showing the analysis results of an acute phase in Embodiment 1, wherein FIG. 1A is a comparison of superoxide content in the right hamstring muscles of each group of rats, FIG. 1B is a comparison of the fluorescence of DHE staining of the right hamstring muscles of each group of rats, FIG. 1C is a comparison of myocardial superoxide content in each group of rats, FIG. 1D a comparison of the fluorescence of myocardial DHE staining in each group of rats, FIG. 1E is a comparison of the number of ADSCs after immunohistochemical staining with anti-human mitochondrial antibodies in the right hamstring muscles of each group of rats, and FIG. 1F is a comparison of the expression of Alu gene in the myocardium of each group of rats after analysis by RT-PCR. * in the figure indicates p<0.05 compared to the control group (WKY); † in the figure indicates p<0.5 compared to the vehicle group treated with vehicle (veh); ‡ in the figure indicates ‡P<0.05 compared to the ADSC group (ADSC). (for each group, N=5)

FIGS. 2A to 2B are analysis results showing the effect of remote transplantation of human ADSC on myocardial ROS levels in the chronic phase in Embodiment 1, wherein FIG. 2A is a comparison of myocardial superoxide content in each group of rats, and FIG. 2B is a comparison of the fluorescence of myocardial DHE staining in each group of rats. * in the figure indicates p<0.05 compared to the control group (WKY); † in the figure indicates p<0.5 compared to the vehicle group treated with vehicle (veh); ‡ in the figure indicates P<0.05 compared to the ADSC group (ADSC). (for each group, N=10)

FIGS. 3A to 3C are showing the analysis results of myocardial STAT3 activity in the chronic phase in Embodiment 1, wherein FIG. 3A is a comparison of the analysis of p-STAT3 (phospho-STAT3) content by Western blotting, FIG. 3B is a comparison of the analysis of the DNA binding activity of STAT3 by ELISA, and FIG. 3C is a quantitative graph of the degree of nuclear translocation of STAT3 after immunohistochemical staining with anti-p(tyr 705)-STAT3 antibody. * in the figure indicates p<0.05 compared to the control group (WKY); † in the figure indicates p<0.5 compared to the vehicle group treated with vehicle (veh); ‡ in the figure indicates P<0.05 compared to the ADSC group (ADSC). (for each group, N=10)

FIGS. 4A and 4B are analysis results showing the effect of remote transplantation of human ADSC on myocardial macrophage phenotype in the chronic phase in Embodiment 1, wherein FIG. 4A is a quantitative comparison of the analysis of the expression of M1 macrophages by immunohistochemical staining in the vehicle group (Veh) and ADSC group (ADSC), and FIG. 4B is a quantitative comparison of the analysis of the expression of M2 macrophages by immunohistochemical staining in the vehicle group (Veh) and ADSC group (ADSC). * in the figure indicates p<0.001 compared to the control group (WKY). (for each group, N=5)

FIGS. 5A to 5E are analysis results showing the effect of remote transplantation of human ADSC on myocardial macrophage phenotype in the chronic phase in Embodiment 1, wherein FIG. 5A is a comparison of IL-6 expression levels in myocardium of each group of rats, FIG. 5B is a comparison of IL-1β expression levels in myocardium of each group of rats, FIG. 5C is a comparison of iNOS expression levels in myocardium of each group of rats, FIG. 5D is a comparison of expression levels of CD206 in myocardium of each group of rats, and FIG. 5E is a comparison of expression levels of IL-10 in myocardium of each group of rats. * in the figure indicates p<0.05 compared to the control group (WKY); † in the figure indicates p<0.5 compared to the vehicle group treated with vehicle (veh); ‡ in the figure indicates P<0.05 compared to the ADSC group (ADSC). (for each group, N=10)

FIGS. 6A to 6D are showing the analysis results showing the effect of remote transplantation of human ADSC on myocardial hypertrophy and fibrosis in the chronic phase in Embodiment 1, wherein, FIG. 6A is a comparison of cardiomyocyte cross-section areas in each group of rats, FIG. 6B is a comparison of expression levels of BNP gene in rat myocardium, FIG. 6C is a quantitative comparison of the fibrosis area after Sirius red staining, and FIG. 6D is a comparison of the results of the analysis of hydroxyproline content. * in the figure indicates p<0.05 compared to the control group (WKY); † in the figure indicates p<0.5 compared to the vehicle group treated with vehicle (veh); ‡ in the figure indicates P<0.05 compared to the ADSC group (ADSC). (for each group, N=5)

FIGS. 7A to 7C are showing the analysis results of the in vitro experiment of Embodiment 2, wherein, FIG. 7A is a comparison of the content of myocardial superoxide in each group, FIG. 7B is a comparison of the STAT3 activation of each group, and FIG. 7C is a comparison of expression levels of IL-10 in each group. * in the figure indicates p<0.05 compared to the ADSC group; † in the figure indicates p<0.5 compared to the BP/ADSC group; ‡ in the figure indicates P<0.05 compared to the BP/ADSC/SIN group. (for each group, N=5)

### DETAILED DESCRIPTION OF THE INVENTION

Hereinafter, the embodiments of the present invention will be described in more detail with reference to different embodiments, so as to make the spirit and content of the present invention more complete and easy to understand. However, those having ordinary skill in the art will appreciate that the present invention is of course not limited by such examples, and other similar or equivalent functions and steps may be utilized to achieve the present invention.

First, a descriptive illustration will be made on specific terms or nouns used in this specification.

Unless otherwise defined in the specification, the meaning of the scientific and technical terms used herein is the same as that understood by those having ordinary skills in the art to which the present invention pertains.

Herein, the term “treatment” or “treating” means partial or complete reduction in severity, delay in progression, and/or inhibition of one or more conditions, abnormalities, and/or signs of disease in the medical condition by performing a prophylactic, curative or palliative treatment that achieves pharmaceutically and/or physiological effects on individuals or patients with certain medical conditions, symptoms, diseases, conditions, or their prior status. The above conditions, diseases, abnormalities and/or medical conditions may be sciatic nerve injuries.

Herein, the term “an effective amount” means the specific amount that can achieve the effect of slowing ventricular hypertrophy or treating ventricular hypertrophy after appropriate dosing period for medical drugs directly or indirectly administrated (administered, administration, or administration) to patients.

Herein, the above “medical drugs” means pharmaceutically active substances capable of inducing a desired pharmaceutical and/or physiological response through local and/or systemic action, typically including compound, formulation, composition, agent, medicine or medicament, or prodrug, derivative or analog etc.

Herein, the term “subject” or “patient” can be used interchangeably with each other. The term “individual” or “patient” refers to an animal that is treatable by the compound and/or method, respectively, including but not limited to, for example, dogs, cats, horses, sheep, pigs, cows, and the like, as well as human, non-human primates. Unless otherwise specified, the “subject” or “patient” may include both male and female genders. Further, it also includes a subject or patient, preferably a human, suitable for receiving treatment with a pharmaceutical composition and/or method of the present invention.

Herein, the numerical values and parameters used to define the scope of the present invention intrinsically inevitably contain standard deviations due to individual test methods, and hence mostly expressed in terms of approximate numerical values, but in the specific embodiments, are related numerical values presented as accurately as possible. The term “about” herein usually means that the actual value falls within the acceptable standard error of the average depending on the consideration of those having ordinary skills in the art to which the present invention pertains. For example, the actual value is within ±10%, ±5%, ±1%, or ±0.5% of a particular value or range.

The disclosure of the present invention may provide a pharmaceutical composition for treating cardiac hypertrophy, comprising at least a stem cell and a pharmaceutically acceptable vehicle, wherein the stem cell is prepared by a pretreatment reaction of reacting with an n-butylidenephthalide (BP). According to the technical idea of the present invention, the concentration of n-butylidenephthalide in the pretreatment reaction is in the range of 7 μg/mL to 40 μg/mL, preferably in the range of 7 μg/mL to 35 μg/mL, more preferably in the range of 7 μg/mL to 30 μg/mL, most preferably in the range of 7 μg/mL to 25 μg/mL. The condition of the pretreatment reaction comprises reacting the stem cell with the n-butylidenephthalide at a temperature range of 20 to 40° C., preferably of 25 to 40° C., more preferably of 30 to 40° C., most preferably of 35 to 40° C. In addition, the reaction time is in the range of 6 hours to 24 hours, preferably of 7 hours to 24 hours, more preferably 8 hours to 24 hours, most preferably 10 hours to 24 hours.

Further, according to the technical idea of the present invention, the stem cell is at least one selected from a group consisting an umbilical cord blood stem cell, a peripheral blood stem cell, a neural stem cell, an epidermal stem cell, a muscle stem cell, an adipose stem cell, a bone marrow stem cell, a corneal stem cell, a liver stem cell, and an intestinal epithelial stem cell, preferably one selected from a group consisting an umbilical cord blood stem cell, a peripheral blood stem cell, a neural stem cell, an adipose stem cell, a bone marrow stem cell, a liver stem cell and an intestinal epithelial stem cell, more preferably one selected from a group consisting an umbilical cord blood stem cell, a peripheral blood stem cell, a neural stem cell, an adipose stem cell, and a bone marrow stem cell, most preferably one selected from a group consisting an umbilical cord blood stem cell, an adipose stem cell and a bone marrow stem cell.

The specific embodiments of the present invention are described below by way of embodiments, but the scope of the present invention is not limited by the embodiments.

Also, the cell culture experiment and the animal experiment carried out in the embodiments of the present invention are made according to Guidelines for Management and Use of Experimental Animals of the Chinese Medical University (License No.: 2018-043), and confirmed by Guidelines for Management and Use of Experimental Animals of U.S. National Institutes of Health (NIH Publication No. 85-23, revised in 1996).

### Separation of Human ADSCs

The human adipose stem cells (ADSCs) used in the embodiments of the present invention are purchased from a commercially available kit (Stempro human ADSC kit; Invitrogen, Carlsbad, Calif., U.S.A.) and supplied by the Gwo Xi Stem Cell Applied Technology Co., Ltd. The ADSCs is cultured in DMEM medium (Invitrogen) containing 10% FBS (Serana) and 1% penicillin/streptomycin (Hyclone), and subcultured using TrypLE Express (Invitrogen). The cells obtained by subculture are regarded as a first generation, and the ADSCs of the 3-5th generation are used in the present embodiment. These ADSCs are identical and do not contain endothelial cells or hematopoietic lineages. Also, these cultured ADSCs have a mesenchymal stem cell phenotype: expressing mesenchymal stem cell marker CD90 (>95%), not expressing hematopoietic markers CD31 and CD45 (<2%).

### Pretreatment

The n-butylidenephthalide (BP) purchased from Alfa Aesar is dissolved in dimethyl sulfoxide to prepare a BP solution having a concentration of 7 μg/ml. Then, 1×10⁶ to 1×10⁷ ADSCs are put into the BP solution for 16 hours to obtain a BP-pretreated ADSCs.

The cells are washed three times with PBS before cell transplantation to remove direct effects from drugs.

### Embodiment 1

The 12-week-old male spontaneously hypertensive rats (SHR) having random cardiac hypertrophy are randomly divided into three groups: a vehicle group, an ADSC group, and a BP/ADSC group, wherein in the vehicle group 30 μl of PBS is injected to the right hamstring muscle of SHR, in the ADSC group 1×10⁶ ADSCs (mixed in 30 μl of PBS) are injected to the right hamstring muscle of SHR, and in the BP/ADSC 1×10⁶ BP-pretreated ADSCs (mixed in 30 μl of PBS) are injected to the right hamstring muscle of SHR. In addition, male Wistar-Kyoto (WKY) rats of the same age and normal blood pressure are selected as a control group.

The analysis for results is made in terms of two parts, i.e., the acute phase and the chronic phase. For the acute phase, the rats are sacrificed 3 days after transplantation of the sample, and the right hamstring muscles (i.e., the injection region) and the heart are removed for analysis. For the chronic phase, the rats are sacrificed 56 days after the transplantation of the sample, and hearts are removed for analysis.

Next, the standard operation method of each test in the present embodiment will be described below.

### Hemodynamic Test

After 8 weeks of feeding, the arterial blood pressure of the conscious rats is measured using a tailcuff system (BP-98A, Softron Beijing, Bejing, China) in a dark room with a temperature of 22° C. in the morning, and the results are recorded.

Then, intraperitoneal injection is performed with Zoletil (50 mg/kg), the rats are lightly anesthetized and subjected to echocardiography using a GE Healthcare Vivid 7 Ultra-sound System (Milwaukee, Wis.) equipped with a 14-MHz probe, and a M-mode echocardiography of the left ventricle (LV) is obtained from the parasternal long-axis view, so as to obtain ventricular septal size, left ventricular end diastolic diameter (LVEDD), left ventricular end systolic diameter (LVESD), left ventricular posterior wall size, and fractional shortening.

Then, the rats are sacrificed to remove the heart, the atrium and the right ventricle are excised, and the left ventricular muscles are washed in cold physiological saline, weighed, and immediately frozen in liquid nitrogen for use.

### Western Blot Analysis of STAT3

Sampling is performed from the above left free ventricular free wall preserved in liquid nitrogen, and antibodies against p-STAT3 (Tyr705) (Cell Signaling Technology, Danvers, Mass., USA), total STAT3 (Santa Cruz Biotechnology) and β-actin (Santa Cruz Biotechnology, Santa Cruz, Calif.) are used. The sample is homogenized, and the protein concentration is determined with the BCA Protein Assay Kit (Pierce, Rockford, Ill.); 20 μg of protein is taken from the sample and separated by 8% SDS-PAGE, then transferred to the nitrocellulose membrane, then incubated with the above antibody; subsequently, antigen-antibody complexes are detected using 5-bromo-4-chloro-3-indolyl-phosphate and nitroblue tetrazolium chloride (Sigma), and the density scanner is used to detect exposure levels in the linear range. The experiment is repeated three times and the results are expressed as an average.

Real-Time RT-PCR Analysis of Human Alu, IL-6, IL-1β, iNOS, CD206, IL-10, and BNP

In the present embodiment, RT-PCR assays are performed on samples obtained from left ventricular muscle using the TaqMan system (Prism 7700 Sequence Detection System, PE Biosystems) to analyze the expression of gene marker for M1 macrophages (CD206, IL-10) and gene marker for M2 macrophages (IL-6, IL-1β, iNOS) in the sample. In addition, since the molecular hallmarker of cardiac hypertrophy is the reactivation of congenital heart disease genes including B-type Natriuretic Peptide (BNP) in adult hearts, RT-PCR analysis of BNP is also performed to confirm gene expression of cardiac hypertrophy.

When RT-PCT is performed, the amplification is carried out for 45 cycles (95° C.° C. (10 seconds)→60° C. (5 seconds)→75° C. (10 seconds)) in the TaqMan system using the primer sequences shown in Table 1 below. Then, a standard curve of the threshold of the threshold cycle relative to the target template is plotted, and the expression level of each gene is obtained after conversion based on the expression of the housekeeping gene cyclophilin.

TABLE 1

| | |
|---|---|
| Alu | sense primer: 5′-CATGGTGAAACCCCGTCTCTA-3′ (SEQ ID: NO. 1) |
| | antisense primer: 5′-GCCTCAGCCTCCCGAGTAG-3′ (SEQ ID: NO. 2) |
| IL-6 | sense primer: 5′-CCAGTTGCCTTCTTGGGACTGATG-3′ (SEQ ID: NO. 3) |
| | antisense primer: 5′-ATTTTCTGACCACAGTGAGGAATG-3′ (SEQ ID: NO. 4) |
| IL-1β | sense primer: 5′-ATGGCAACTGTCCCTGAACTCAACT-3′ (SEQ ID: NO. 5) |
| | antisense primer: 5′-CAGGACAGGTATAGATTCAACCCCTT-3′ (SEQ ID: NO. 6) |
| iNOS | sense primer: 5′-TCACCTTCGAGGGCAGCCGA-3′ (SEQ ID: NO. 7) |
| | antisense primer: 5′-TCCGTGGCAAAGCGAGCCAG-3′ (SEQ ID: NO. 8) |
| CD206 | sense primer: 5′-TGGGTTTGCTGAAGAAGAGAA-3′ (SEQ ID: NO. 9) |
| | antisense primer: 5′-CATGTGATAAGTGACAAATGCTTG-3′ (SEQ ID: NO. 10) |
| IL-10 | sense primer: 5′-GGTTGCCAAGCCTTGTCAGAA-3′ (SEQ ID: NO. 11) |
| | antisense primer: 5′-GCTCCACTGCCTTGCTTTTATT-3′ (SEQ ID: NO. 12) |
| cyclophilin | sense primer: 5′-ATGGTCAACCCCACCGTGTTCTTCG-3′ (SEQ ID: NO. 13) |
| | antisense primer: 5′-CGTGTGAAGTCACCACCCTGACACA-3′ (SEQ ID: NO. 14) |
| BNP | sense primer: 5′-AGAGAGCAGGACACCATC-3′ (SEQ ID: NO. 15) |
| | antisense primer: 5′-AAGCAGGAGCAGAATCATC-3′ (SEQ ID: NO. 16) |

### Immunohistochemical Staining Analysis of Human Mitochondria, Nitrotyrosine,

CD68, iNOS, and IL-10, and Superoxide Detection Thereof

The hamstring muscle and myocardial samples obtained after the third day of injection are used to confirm whether the ADSCs had been successfully implanted into the tissue. Then, the tissue sample are frozen by using 2-methylbutane, followed by embedding in the optimum cutting temperature (OCT) compound (Tissue-Tek, Torrance), and sectioned at 5 μm on a microtome. The sections are washed with PBTx (PBS containing 0.1% Triton X-100), and blocked for 1 hour at 37° C. with PBTx containing 1% BSA (Amresco) and 1.5% normal goat serum (Vector laboratories). Then, the section are incubated overnight with primary antibodies such as anti-human mitochondria (1:100, Chemicon International) and anti-sarcomeric alpha-actinin antibodies (1:100, Abcam, Cambridge, UK) at 4° C.

Also, in order to evaluate whether superoxide is produced in cardiomyocytes, OCT-embedded tissue is used to react with DHE. Generation of superoxide production in cardiomyocytes is evaluated using in situ dihydrochloride (DHE; Invitrogen Molecular Probes, Eugene, Oreg., USA) fluorescence. Paraffin-embedded tissues (5 μm) are soaked in DHE-containing PBS (10 mM) and kept in a humidified container at room temperature in the dark for 30 minutes; then, detection of red fluorescent signals through images shows that the tissue produces superoxide radicals, and a report of a density of random images per square millimeter is output. To minimize interference from non-specific DHE oxidation products, excitation is performed in the wavelength range of 480 nm to 405 nm and a red fluorescence is detected.

Besides, after the 56th day of sample implantation, in order to confirm the shift of macrophage polarization, the left ventricular muscle of each group of rats is analyzed for M1 macrophages and M2 macrophages by immunohistochemical staining.

The tissue is placed in the frozen embedding agent (OCT), put into for freezing and cooling to maintain the tissue type, reacted with antibodies against CD68 (markers for all macrophages; Abcam, Cambridge, Mass.), iNOS (markers of M1; Cell Signaling Technology, Danvers, Mass., USA), and IL-10 (markers of M2c; R& D systems, Abingdon, UK) after cryosectioning, and the antibody that is directly conjugated with the same isotype is used as a negative control group. The target density on the trajectory is calculated by a computerized planar method (Image Pro Plus, Media Cybernetics, Silver Spring, Md.). At 400× magnification, 10 domains are randomly selected to qualitatively evaluate the target density, expressed as the ratio of the marked area to the total area.

Morphometry of Myocyte Size and Cardiac Fibrosis

Since cardiac hypertrophy is a combination of reactive fibrosis and cardiomyocyte hypertrophy, the size of cardiomyocytes is measured in the present embodiment in order to avoid interference of other nonmyocytes for analysis of cardiac hypertrophy caused by only measurement of the myocardial weight.

After the 56th day of sample implantation, the left ventricular muscle section of the rat is fixed by 10% formalin and embedded in paraffin, each section being stained with hematoxylin and eosin. The sampling position for the section is in the middle of the left ventricular muscle, so as to exclude differences in myocardial cell size in different regions of the left ventricular muscle. Then, in order to maintain consistency in the results of the analysis, cardiomyocytes that are perpendicular to the section plane and have clearly visible nuclei and cell membranes with clear and unbroken cell contours is selected for measurement of the cross-sectional area of cardiomyocytes.

The measurement method is to use the computerized planar method (Image Pro Plus, Media Cybernetics, Silver Spring, Md.): at 400× magnification, 100 cardiomyocytes are selected from digital images in the image analysis system for analysis by an operator who does not know the experimental processing.

In addition, the analysis for cardiac fibrosis type is performed by staining paraffin-embedded sections (thickness of 5 μm) of the left ventricular coronary site with aniline blue and collagen-specific staining Sirius Red (Sirius Red F3BA; Pfaltz & Bauer, Stamford, Conn.). The interstitial collagen fraction is determined by quantitative morphometry of the sections stained with Sirius red with an automatic image analyzer (Image Pro Plus, CA). These values are evaluated by at least 2 investigators in a single-blind manner. At 400× magnification, 10 domains are randomly selected to qualitatively evaluate the density of the marked regions. This value is expressed as the ratio of the marked area to the total area.

### Laboratory Measurement

Tissue collagen results are confirmed by hydroxyproline assay adapted from Stegemann and Staller. The sample from the free wall of the left myocardium is taken out and immediately placed in liquid nitrogen and stored at −80° C. until the hydroxyproline content is measured. The results are calculated based on the hydroxyproline content per tissue weight.

In order to evaluate the DNA binding activity of STAT3, myocardial homogenates are prepared and subjected to ELISA analysis using the commercially available kit TransAM STAT3 Transcription Factor Assay Kit (Active Motif) and its analytical method.

The activity of IL-10 in myocardium is measured for M2c macrophages. The myocardial tissues from the free wall of the left ventricle are homogenized in extraction buffer (50 mM potassium phosphate buffer, pH 7.0; 1 mM EDTA; 1 mM ethylene glycol tetraacetic acid; 0.2 mM phenylmethanesulfonyl fluoride; 1 μg/mL pepstatin; 0.5 μg/mL leupeptin; 10 mM NaF; 2 mM Na3VO4; and 10 mM β-mercaptoethanol) and centrifuged at 14,000 g for 30 minutes at 4° C. Then, the fraction of membrane-bound IL-10 in the myocardium is measured using the commercially available ELISA kit (R&D Systems).

The chemiluminescence enhanced by chemiluminescence (5 μM bis-N-methylacridinium nitrate, Sigma, St. Louis, Mo.) is used to detect the generation of myocardial superoxide, as described above. Specific chemiluminescence signals are calculated after subtracting background activity, and expressed in milligrams per minute (cpm/mg).

### Statistical Analysis

The results of the analysis are expressed as mean±SD. The statistical analysis is performed using the SPSS statistical analysis software package (SPSS, version 19.0, Chicago, Ill.). The differences between the rats in each group are tested by ANOVA. In the case of significant effects, measurements between groups are compared to Bonferroni's correction. P value of <0.05 represents that the statistic has significance.

Then, the test results of each analysis will be described below.

### Characteristics of Human ADSCs

After analysis by flow cytometry, it can be known that ADSCs is highly positive for CD73, CD90, and CD105, and does not express for CD14, CD19, CD34, CD45, and HLA-DR. Therefore, the phenotype of human ADSCs cultured in the embodiments of the present invention is CD73pos/CD90pos/CD105 pos/CD14neg/CD19neg/CD34neg/CD45neg/HLA-DRneg.

These ADSCs are homogeneous and do not contain endothelial cells or hematopoietic lineages, and the pretreatment of ADSC with BP for 16 hours prior to transplantation may not result in significant changes in cell markers.

### Analysis Results for the Acute Phase (3 Days)

The analysis samples of the acute phase are taken from the right hamstring muscles (transplantation region) and left ventricular muscle of each group of rats 3 days after the transplantation of the samples.

### Effects of ADSC Transplantation on Superoxide in Right Hamstring Muscle and Myocardium

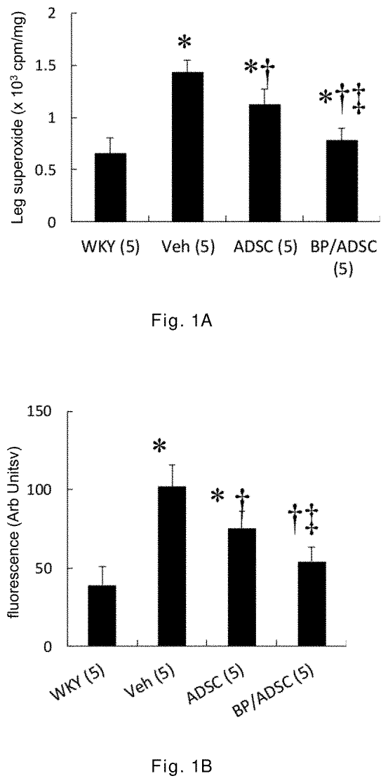

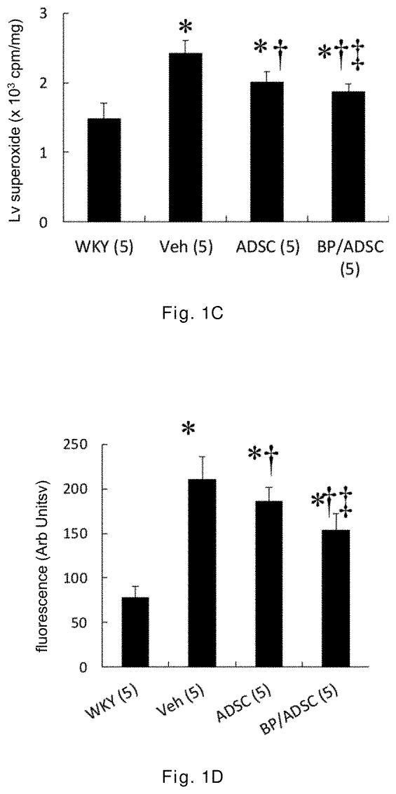

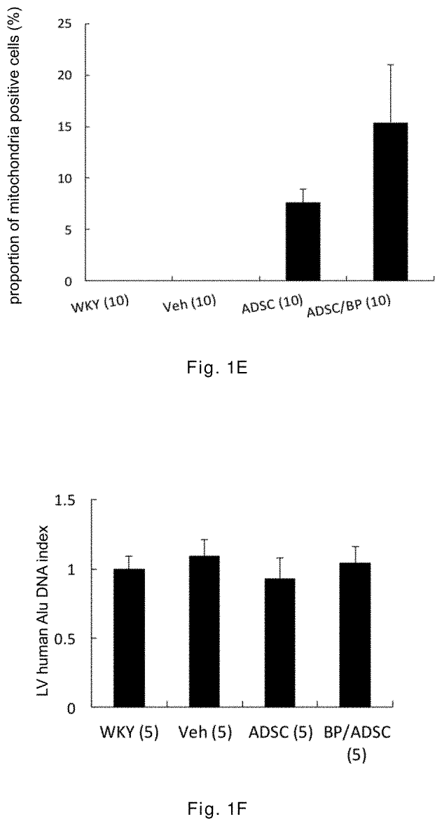

The right hamstring muscle and left ventricular muscle samples of each group of rats are subjected to chemiluminescence enhanced by gloss agents and detected for specific chemiluminescence signals from superoxide. The results obtained are shown in Table 2, and plotted as FIGS. 1A and 1C, respectively.

Further, the right hamstring muscle and left ventricular muscle samples of each group of rats are subjected to DHE fluorescence staining, and photographed by fluorescence microscope and analyzed for the amount of red fluorescence of the samples. The results obtained are recorded in Table 2, and plotted as FIGS. 1B and 1D, respectively.

Also, in order to quantify the survival rate of human ADSCs implanted in rats, the right hamstring muscle samples of each group of rats are stained with anti-human mitochondria antibody to analyze the proportion of mitochondrial positive cells in the samples, which are then recorded in Table 2 and plotted as FIG. 1E.

Since the presence of stem cells may not be detected by anti-human mitochondrial antibody staining in the left ventricular muscle samples of each group of rats after 3 days of transplantation, the expression of Alu gene in ventricular muscle samples is analyzed by PT-PCR (the primer sequence used is shown in Table 3) in order to avoid the problem that immunohistochemistry in vivo may not be sensitive enough to detect a small number of cells. The results of the analysis are recorded in Table 2, and plotted as FIG. 1F.

TABLE 2

| | | control group | vehicle group | ADSC group | BP/ADSC group |
|---|---|---|---|---|---|
| acute phase (3 days) | | (WKY) | (Veh) | (ADSC) | (BP/ADSC) |
| right | superoxide | 0.65 ± 0.15 | 1.43 ± 0.12 | 1.12 ± 0.15 | 0.78 ± 0.12 |
| hamstring | (×10³ cpm/mg) | | | | |
| muscle | fluorescence | 39 ± 12 | 102 ± 14 | 75 ± 11 | 54 ± 9 |
| | (Arb Unitsv) | | | | |
| | proportion of | 0 | 0 | 7.6 ± 1.3 | 15.4 ± 5.6 |
| | mitochondria positive | | | | |
| | cells (%) | | | | |
| left | superoxide | 1.48 ± 0.23 | 2.43 ± 0.18 | 2.01 ± 0.15 | 1.87 ± 0.12 |
| ventricular | (×10³ cpm/mg) | | | | |
| myocardium | fluorescence | 78 ± 13 | 211 ± 25 | 186 ± 16 | 154 ± 18 |
| | (Arb Unitsv) | | | | |
| | Performance level of | 1 ± 0.09 | 1.09 ± 0.12 | 0.93 ± 0.12 | 1.04 ± 0.12 |
| | human Alu DNA | | | | |

From the results of the specific chemiluminescence signal from the right hamstring muscle samples shown in FIG. 1A, it can be known that the content of superoxide in the vehicle group is significantly higher than that in the control group (P<0.01); compared with the vehicle group, the superoxide content of the ADSC group and the BP/ADSC group are significantly decreased, wherein the decrease amplitude in the BP/ADSC group is the largest. In addition, the corresponding results may also be observed from the results of DHE fluorescence staining analysis shown in FIG. 1B.

Similarly, from the results of the specific chemiluminescence signal from the left ventricular muscle samples shown in FIG. 1C, it can be known that the content of superoxide in the vehicle group is significantly higher than that in the control group (P<0.01); compared with the vehicle group, the superoxide content of the ADSC group and the BP/ADSC group are significantly decreased, wherein the decrease amplitude in the BP/ADSC group is the largest. In addition, the corresponding results may also be observed from the results of DHE fluorescence staining analysis shown in FIG. 1D.

Specifically, for the decreasing effect of superoxide in the right hamstring muscles, the ADSC group decreases by approximately 21.68% and the BP/ADSC group decreases by approximately 45.45% compared to the vehicle group, indicating that the BP/ADSC group has a decreasing effect of up to 2.1 times that of ADSC group. Secondly, for the decreasing effect of superoxide in the left ventricular myocardium, the ADSC group decreases by approximately 17.28% and the BP/ADSC group decreases by approximately 23.05% compared to the vehicle group, indicating that the BP/ADSC group has a decreasing effect of up to 1.33 times or more that of ADSC group. Therefore, from the above results, it can be known that implantation of ADSC or BP/ADSC in vivo may effectively decrease the superoxide content increased by hypertension, but the decreasing effect of BP/ADSC is much higher than that of ADSC; and, in terms of administration, for BP/ADSC group, implantation from the heart is not required as long as having implantation from a remote location (such as the right hamstring muscle), so this has an excellent effect of reducing the risk of patients.

From the results of staining analysis of anti-human mitochondrial antibodies shown in FIG. 1E, it can be known that as observed from the region in which the sample is implanted (right hamstring muscle), the proportion of mitochondria-positive cells in the ADSC group is 7.6±1.3%, but the proportion of mitochondria-positive cells in the BP/ADSC group increases to 15.4±5.6% with a significant increase (P<0.05), which indicated that the pretreatment with ADSC through BP may effectively increase the survival rate of ADSCs in muscle.

In addition, the results of the Alu gene expression in the left ventricular muscle samples shown in FIG. 1F, it can be known that in the left ventricular muscle samples of the control and vehicle groups, ADSC group and BP/ADSC group, the Alu gene expressions are almost equal without significant differences, indicating that the remotely transplanted ADSCs (e.g., right hamstring muscles) does not significantly migrate from the injection site to the heart.

### Analysis for the Chronic Phase (56 Days)

The analysis samples of the chronic phase are taken from the left ventricular muscle of each group of rats 56 days after the transplantation of the samples. The hemodynamics data of each group are shown in Table 3.

TABLE 3

| chronic phase | control | vehicle | ADSC | BP/ADSC |
|---|---|---|---|---|
| (56 days) | group | group | group | group |
| Rat breed | WKY | SHR | SHR | SHR |
| number of rats | 10 | 10 | 10 | 10 |
| average weight (g) | 402 ± 16 | 302 ± 12* | 311 ± 18* | 305 ± 15* |
| heartbeat (bpm) | 384 ± 12 | 388 ± 15 | 402 ± 18 | 392 ± 21 |
| systolic blood | 98 ± 7 | 214 ± 11* | 201 ± 14* | 209 ± 15* |
| pressure (mmHg) | | | | |
| left ventricular | 252 ± 21 | 395 ± 31 | 349 ± 28*† | 329 ± 22*†‡ |
| weight/tibia | | | | |
| length (mg/cm) | | | | |

*indicates p < 0.05 compared to the ADSC group;
†indicates p < 0.5 compared to the BP/ADSC group;
‡indicates ‡P < 0.05 compared to the BP/ADSC/SIN group.

The SHR group has a similar weight basis before cell transplantation. From the results of Table 1, it can be known that the body weight of the vehicle group, the ADSC group, and the BP/ADSC group are similar after 56 days of cell transplantation, indicating that the subject's body weight does not change significantly due to cell transplantation.

In addition, for the ratio of the left ventricle weight to the tibia length, the vehicle group increases by 69% compared with the control group, indicating that the vehicle group has symptoms of cardiac hypertrophy; compared with the vehicle group, the ADSC group and the BP/ADSC group decrease by 11.6% and 16.7%, respectively, indicating that ADSC and BP/ADSC may effectively reduce cardiac hypertrophy caused by hypertension, and BP/ADSC has a decreasing effect of up to 1.44 times that of ADSC.

Also, by comparing statistics of other hemodynamic parameters such as blood pressure and heartbeat, it can be found that there are no significant differences among tissues in the vehicle group, the ADSC group, and the BP/ADSC group, indicating that ADSC's treatment for cardiac hypertrophy is via nonhemodynamic effects. Further, rats in each group have no local infection or inflammation around the site of injection.

Effects of Remote Transplantation of Human ADSC on Cardiac ROS and STAT3 Activation

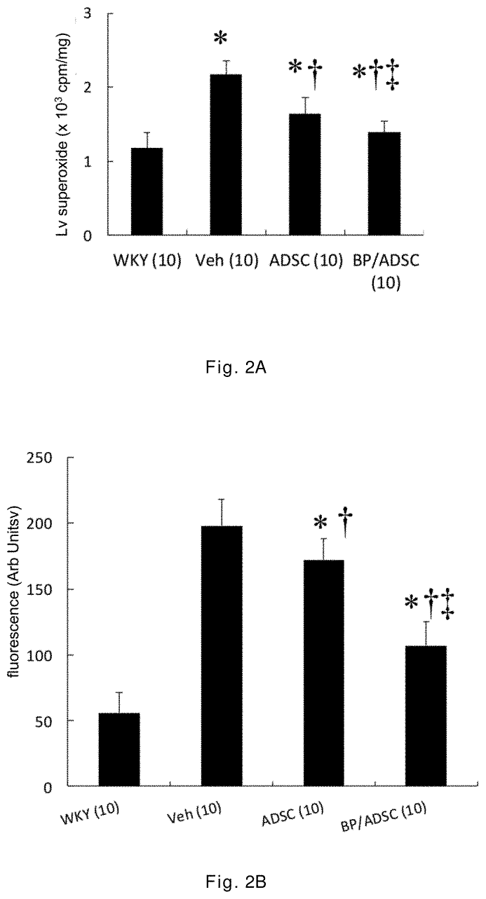

The left ventricular muscle samples of each group of rats are subjected to chemiluminescence enhanced by gloss agents and detected for specific chemiluminescence signals from superoxide. The results obtained are shown in Table 4, and plotted as FIG. 2A.

Further, the right hamstring muscle and left ventricular muscle samples of each group of rats are subjected to DHE fluorescence staining, and photographed by fluorescence microscope and analyzed for the amount of red fluorescence of the samples. The results obtained are recorded in Table 4, and plotted as FIG. 2B.

TABLE 4

| chronic | control | vehicle | ADSC | BP/ADSC |
|---|---|---|---|---|
| phase | group | group | group | group |
| (56 days) | (WKY) | (Veh) | (ADSC) | (BP/ADSC) |
| myocardial superoxide | 1.18 ± 0.21 | 2.17 ± 0.19 | 1.64 ± 0.22 | 1.39 ± 0.15 |
| (×10³ cpm/mg) | | | | |
| fluorescence | 56 ± 15 | 198 ± 20 | 172 ± 16 | 107 ± 18 |
| (Arb Unitsv) | | | | |

From the results of the specific chemiluminescence signal from the left ventricular muscle samples shown in FIG. 2A, it can be known that compared with the vehicle group, the superoxide content (P<0.001) of the ADSC group and the BP/ADSC group are significantly decreased, wherein the decrease amplitude of superoxide content in the BP/ADSC group is the largest as compared with ADSC group. Specifically, in the analysis for the chronic phase, for the decreasing effect of superoxide in the left ventricular muscles, the ADSC group decreases by approximately 24.42% and the BP/ADSC group decreases by approximately 35.94% compared to the vehicle group, indicating that the BP/ADSC group has a decreasing effect of up to 1.5 times that of ADSC group. In addition, from the graph of DHE fluorescence staining analysis shown in FIG. 2B, it can be known that the fluorescence intensity ratio of the vehicle group is significantly higher than that of the control group, but compared with the vehicle group, the fluorescence intensity of the ADSC group and the BP/ADSC group significantly decreases, wherein the decreasing amplitude of fluorescence intensity of the BP/ADSC group is the largest as compared with the ADSC group. This result corresponds to the specific chemiluminescent signal result.

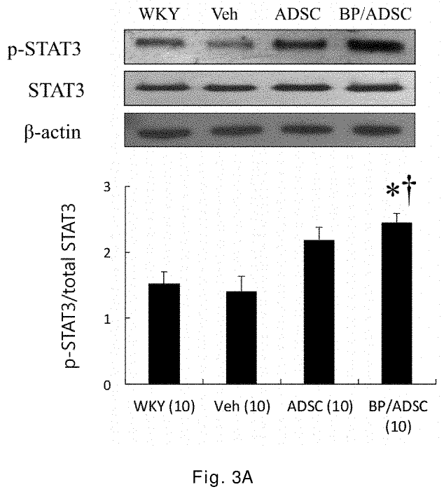

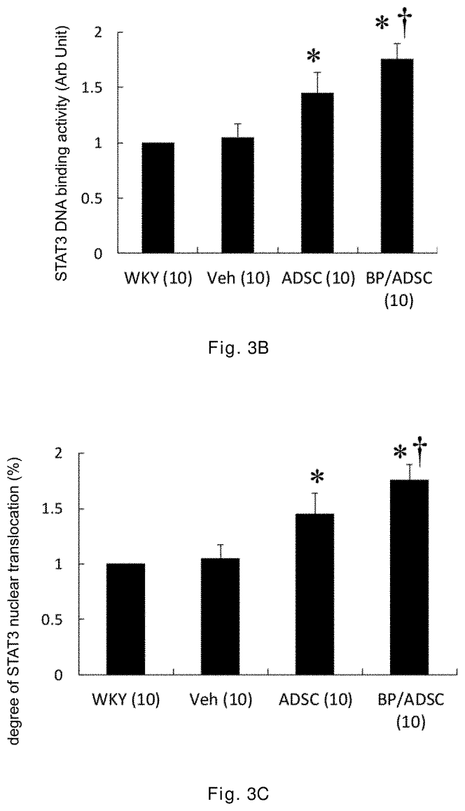

Further, the results of the Western blot analysis are shown in Table 5 and FIG. 3A below. From the results of FIG. 3A, it can be known that the proportion of p-STAT3 (phospho-STAT3) to total STAT3 (p-STAT3/total STAT3) in the vehicle group is significantly lower than that of the control group (P<0.05), and the ADSC group may increase the proportion of p-STAT3 to 156±21% (P<0.01), indicating that the use of ADSC may effectively promote activation. In addition, for the effect of p-STAT3 activation, the BP/ADSC group (2.45±0.14%) is approximately 1.12 times as high as the ADSC group (2.19±0.19%).

Also, the results of the detection of DNA-binding activity of STAT3 using ELISA method are shown in Table 5 and FIG. 3B below. From the results of FIG. 3B, it can be known that the ADSC group (1.45±0.19) increases by about 1.38 times compared with the vehicle group (1.05±0.12), and the BP/ADSC group (1.76±0.14) increases by about 1.68 times compared with the vehicle group (1.05±0.12), indicating that the DNA binding activity of STAT3 in the BP/ADSC group is significantly higher than that in the ADSC group, which is 1.22 times higher than that in the ADSC group. This result corresponds to the results obtained by Western blot analysis.

Further, in order to evaluate the degree of STAT3 activation in left ventricular muscle in each group of rats, immunohistochemical staining is performed with anti-p(tyr 705)-STAT3 antibody, and the degree of nuclear translocation of STAT3 is analyzed from fluorescence micrographs. The quantified data obtained is recorded in Table 5 and plotted as FIG. 3C.

From the results of FIG. 3C, the degree of STAT3 nuclear translocation in the BP/ADSC group is significantly higher than that in the vehicle group and the ADSC group. The results of this analysis correspond to the results of the Western blot analysis and ELISA method described above.

TABLE 5

| chronic | control | vehicle | ADSC | BP/ADSC |
|---|---|---|---|---|
| phase | group | group | group | group |
| (56 days) | (WKY) | (Veh) | (ADSC) | (BP/ADSC) |
| p-STAT3/total STAT3 | 1.52 ± 0.18 | 1.4 ± 0.24 | 2.19 ± 0.19 | 2.45 ± 0.14 |
| DNA binding activity of | 1 ± 0 | 1.05 ± 0.12 | 1.45 ± 0.19 | 1.76 ± 0.14 |
| STAT3 (Arb Unit) | | | | |
| degree of STAT3 nuclear | 1 ± 0 | 1.05 ± 0.12 | 1.45 ± 0.19 | 1.761 ± 0.14 |
| translocation (%) | | | | |

Effect of Remote Transplantation of Human ADSC on Cardiac Acrophages Skewing Toward a M2 Phenotype

In order to explore the interaction of human ADSCs with host macrophages in the myocardium, the immunohistochemical staining is used to identify specific markers for different phenotypes for analysis. The phenotype after polarization of macrophage may be mainly divided into M1 and M2, wherein M1 macrophages are pro-inflammatory, and M2 macrophages may promote inflammation and reduce inflammation.

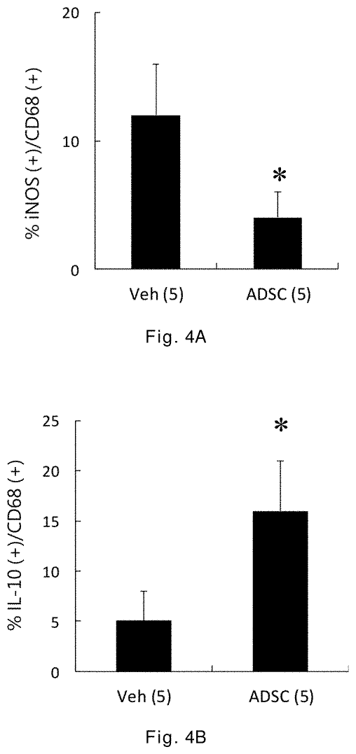

The results of immunohistochemical staining show that CD68+ macrophage is infiltrated. The proportion of iNOS-expressing CD68+ macrophage is significantly reduced in the ADSC group compared to the vehicle group (12±4% for the vehicle group, 4±2% for the ADSC group, P<0.05), as shown in Table 6 and FIG. 4A; also, the proportion of IL-10-expressing CD68+ macrophage is significantly increased in the ADSC group compared to the vehicle group (5±3% for the vehicle group, 16±5% for the ADSC group, P<0.05), as shown in Table 6 and FIG. 4B.

TABLE 6

| | vehicle group | ADSC group |
|---|---|---|
| chronic phase (56 days) | (Veh) | (ADSC) |
| iNOS (+)/CD68 (+)(%) | 12 ± 4 | 4 ± 2 |
| IL-10 (+)/CD68 (+)(%) | 5 ± 3 | 16 ± 5 |

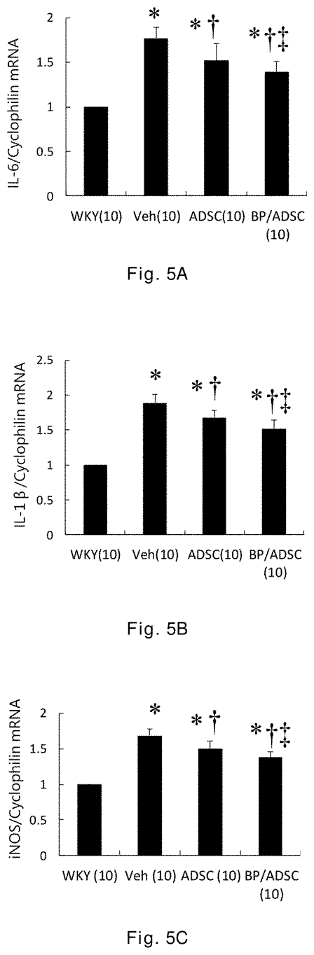

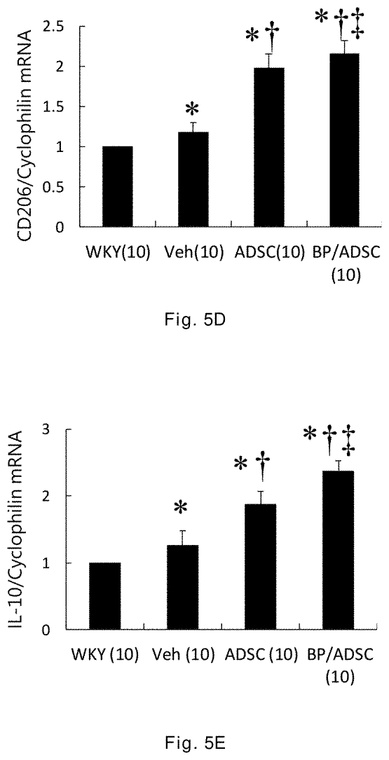

In addition, in order to further confirm the macrophage phenotypic transition, expression levels of the M1 macrophage mRNA (IL-6, IL-1β, and iNOS) and the M2 macrophage mRNA (CD206, and IL-10) in left ventricular muscle of each group of rats are analyzed by RT-PCR. The results are shown in Table 7, and are plotted in FIGS. 5A to 5E.

TABLE 7

| chronic phase | control group | vehicle group | ADSC group | BP/ADSC group |
|---|---|---|---|---|
| (56 days) | (WKY) | (Veh) | (ADSC) | (BP/ADSC) |
| IL-6/Cyclophilin | 1 ± 0 | 1.77 ± 0.12 | 1.52 ± 0.19 | 1.39 ± 0.12 |
| mRNA | | | | |
| IL-1 | 1 ± 0 | 1.89 ± 0.12 | 1.68 ± 0.1 | 1.52 ± 0.12 |
| β/Cyclophilin | | | | |
| mRNA | | | | |
| iNOS/Cyclophilin | 1 ± 0 | 1.69 ± 0.09 | 1.5 ± 0.11 | 1.38 ± 0.08 |
| mRNA | | | | |
| CD206/Cyclophilin | 1 ± 0 | 1.18 ± 0.12 | 1.98 ± 0.18 | 2.16 ± 0.16 |
| mRNA | | | | |
| IL-10/Cyclophilin | 1 ± 0 | 1.26 ± 0.22 | 1.88 ± 0.19 | 2.38 ± 0.15 |
| mRNA | | | | |

From the results of Table 7 and FIGS. 5A to 5E, it can be known that for the results of analysis of mRNA (IL-6, IL-1β, and iNOS) of the M1 macrophages, in the ADSC group, the expression level of IL-6 is 1.52±0.19 that is about 14.12% lower than that in the vehicle group (1.77±0.12), the expression level of IL-1β is 1.68±0.1 that is about 11.11% lower than that of the vehicle group (1.89±0.12), and the expression level of iNOS is 1.5±0.11 that is about 11.24% lower than that of the vehicle group (1.69±0.09); also, in the BP/ADSC group, the expression level of IL-6 is 1.39±0.12 that is about 21.47% lower than that of the vehicle group (1.77±0.12), the expression level of IL-1β is 1.52±0.12 that is about 20.58% lower than that of the vehicle group (1.89±0.12), and the expression level of iNOS is 1.38±0.08 that is about 18.34% lower than that of the vehicle group (1.69±0.09).

Further, in the comparison of the expression level of IL-6, the decreasing degree in the BP/ADSC group is about 1.52 times that of the ADSC group, in the comparison of the expression level of IL-1β, the decreasing degree in the BP/ADSC group is about 1.85 times that of the ADSC group, and in the comparison of the expression level of iNOS, the decreasing degree in the BP/ADSC group is about 1.63 times that of the ADSC group, indicating that the BP/ADSC group is more effective than the ADSC group in decreasing the content of macrophages M1, thereby reducing the chance of inflammation.

In addition, for the results of analysis of mRNA (CD206 and IL-10) of the M2 macrophages, in the ADSC group, the expression level of CD206 is 1.98±0.18 that is 1.68 times higher than that in the vehicle group (1.18±0.12), and the expression level of IL-10 is 1.88±0.19 that is 1.49 times higher than that in the vehicle group (1.26±0.22); also, in the BP/ADSC group, the expression level of CD206 is 2.16±0.16 that is 1.83 times higher than that in the vehicle group (1.18±0.12), and the expression level of IL-10 is 2.38±0.15 that is 1.89 times higher than that in the vehicle group (1.26±0.22).

Further, in the comparison of the expression level of CD206, the increasing degree in the BP/ADSC group is about 1.1 times that of the ADSC group, and in the comparison of the expression level of IL-10, the increasing degree in the BP/ADSC group is about 1.27 times that of the ADSC group, indicating that the BP/ADSC group is more effective than the ADSC group in increasing the content of M2 macrophages, thereby promoting the regression of inflammation and reducing the inflammatory process.

Effect of Remote Transplantation of Human ADSC on Cardiac Hypertrophy and Fibrosis

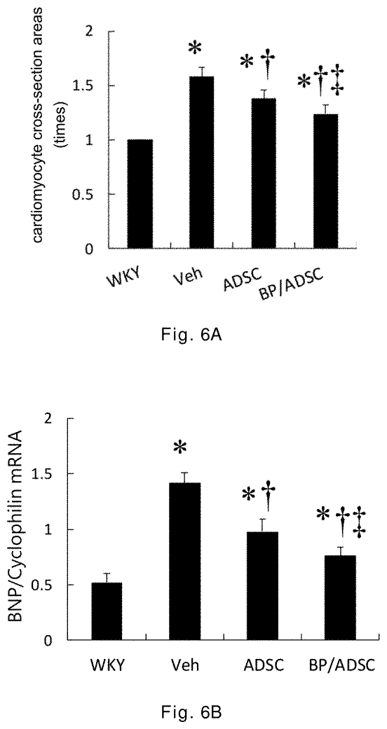

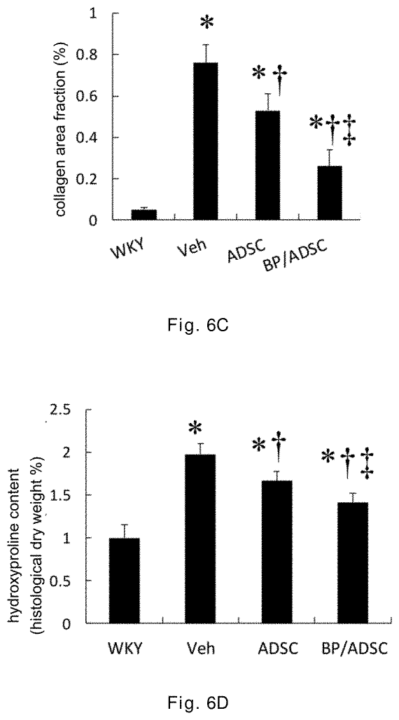

The ratio of the cardiomyocyte cross-section area of the vehicle group, the ADSC group, and the BP/ADSC group to the control group is shown in Table 8 and FIG. 6A. From the results of FIG. 6A, it can be known that the cardiomyocytes in the vehicle group are significantly larger than those in the control group, while the cardiomyocyte cross-section areas in the ADSC group and BP/ADSC are smaller than those in the vehicle group. More specifically, the ratio of cardiomyocyte cross-section areas in the ADSC group is 1.38 times, which is about 13% lower than that in the vehicle group, and the ratio of cardiomyocyte cross-section areas in the BP/ADSC group is 1.24 times, which is about 22% lower than that in the vehicle group. In addition, from these results, it can be known that the effect of reducing the cardiomyocyte cross area in the BP/ADSC group is better than that in the ADSC group, which is about 1.69 times that in the ADSC group.

Also, the results of expression of BNP gene mRNA (a marker of pathological cardiac hypertrophy) in the left ventricula muscle of each group of rats analyzed by RT-PCR are shown in Table 8 and FIG. 6B. From the results of FIG. 6B, it can be known that the expression of BNP gene in the vehicle group is 2.73 times higher than that in the control group (P<0.0001), and the expressions of BNP gene in the ADSC group and the BP/ADSC group decreases as compared with that in the vehicle group. More specifically, the expression of BNP gene in the ADSC group is 0.98±0.11 that is about 31% lower than that in the vehicle group, and the expression of BNP gene in the BP/ADSC group is 0.76±0.08 times, which is about 46% lower than that in the vehicle group. In addition, from these results, it can be known that the BP/ADSC group is better than the ADSC group in suppressing the expression of BNP gene, which is 1.48 times higher than that in the ADSC group. This result is consistent with the results of the aforementioned histology.

Further, the results of analysis of collagen area fraction and the results of analysis of hydroxyproline content performed by picrosirius (Sirius red) staining are shown in Table 8, and are plotted as FIGS. 6C and 6D, respectively.

From the results of analysis of collagen area fraction, it can be known that the collagen area fraction in the ADSC group is 0.53±0.08% that decreases by about 30% as compared with that in the vehicle group, and the collagen area fraction in the BP/ADSC group is 0.26±0.08% that decreases by about 66% as compared with that in the vehicle group, and the decreasing degree in the BP/ADSC group is about 2.2 times that in the ADSC group.

In addition, from the results of analysis of hydroxyproline content, it can be known that the hydroxyproline content in the ADSC group is 1.67±0.11% that decreases by about 16% as compared with that in the vehicle group, the hydroxyproline content in the BP/ADSC group is 1.42±0.1% that decreases by about 28% as compared with that in the vehicle group, and the decreasing degree in the BP/ADSC group is about 1.75 times that in the ADSC group. The trend of these results is also consistent with that of the collagen area fraction, indicating that the BP/ADSC group may significantly reduce cardiac fibrosis as compared with the ADSC group.

TABLE 8

| chronic | control | vehicle | ADSC | BP/ADSC |
|---|---|---|---|---|
| phase | group | group | group | group |
| (56 days) | (WKY) | (Veh) | (ADSC) | (BP/ADSC) |
| cardiomyocyte | 1 ± 0 | 1.58 ± 0.09 | 1.38 ± 0.08 | 1.24 ± 0.08 |
| cross-section area (times) | | | | |
| BNP/Cyclophilin mRNA | 0.52 ± 0.08 | 1.42 ± 0.09 | 0.98 ± 0.11 | 0.76 ± 0.08 |
| collagen area fraction (%) | 0.05 ± 0.01 | 0.76 ± 0.09 | 0.53 ± 0.08 | 0.26 ± 0.08 |
| hydroxyproline content | 1 ± .15 | 1.98 ± 0.12 | 1.67 ± 0.11 | 1.42 ± 0.1 |
| (histological dry weight %) | | | | |

Analysis of Results of Echocardiography

The results of echocardiographic detection are shown in Table 9, and from the results of Table 9, it can be known that compared to the control group, the heart in the vehicle group shows structural changes, such as increased interventricular septum and size of the posterior wall of the left ventricle, indicating the results that are consistent with that of cardiac hypertrophy.

In addition, whether the use of ADSC for the treatment of cardiac hypertrophy will lead to increased wall stress and cardiac dysfunction is confirmed by echocardiography. From the left ventricular diastolic dimension (LVEDD), left ventricular systolic (LVESD) size, and fractional shortening shown in Table 9, it can be known that there are no significant differences in the statistical results among the vehicle group, the ADSC group, and the BP/ADSC group, indicating that the treatment of cardiac hypertrophy using ADSC may not result in increased wall stress and cardiac dysfunction.

TABLE 9

| | control | vehicle | ADSC | BP/ADSC |
|---|---|---|---|---|
| | group | group | group | group |
| interventricular septum | 1.6 ± 0.1 | 1.8 ± 0.1* | 1.7 ± 0.1* | 1.7 ± 0.1* |
| (mm) | | | | |
| thickness of the posterior | 1.6 ± 0.1 | 1.8 ± 0.1* | 1.7 ± 0.1* | 1.6 ± 0.1 |
| wall of the left ventricle | | | | |
| (mm) | | | | |
| LVEDD (mm) | 6.0 ± 0.2 | 6.1 ± 0.3 | 6.1 ± 0.2 | 6.0 ± 0.3 |
| LVESD (mm) | 3.5 ± 0.2 | 3.6 ± 0.2 | 3.5 ± 0.3 | 3.5 ± 0.3 |
| fractional shortening (%) | 42 ± 3 | 41 ± 3 | 43 ± 3 | 42 ± 4 |

*indicates p < 0.05 compared to the ADSC group;

### Embodiment 2

From the results of Embodiment 1, it can be known that the ADSCs pretreated with BP may increase significantly the macrophage M2 phenotype in the myocardium, but the mechanisms involved are unclear. In order to confirm the importance of BP intervention for ROS/STAT3 signaling during macrophage polarization, an in vitro assay is performed using 3-morpholinosydnonimine (SIN-1, peroxynitrite generator) or S3I-201 (a STAT3 inhibitor, 3-(2,4-dichlorophenyl)-4-(1-methyl-1H-indol-3-yl)-1Hpyrrole-2,5-dione, Calbiochem, La Jolla, Calif., USA).

Similarly, the 12-week-old male rats with spontaneously hypertensive (SHR) having random cardiac hypertrophy are randomly divided into four groups: an ADSC group, a BP/ADSC group, a BP/ADSC/SIN group, and a BP/ADSC/S3I group, wherein in the ADSC group 1×10⁶ of ADSCs (mixed in 30 μl of PBS) are injected to the right hamstring muscle of SHR, but in the BP/ADSC group, the BP/ADSC/SIN group, and the BP/ADSC/S3I, 1×10⁶ADSCs pretreated with BP (mixed in 30 μl of PBS) are injected to the right hamstring muscle of SHR.

The rats are sacrificed and the hearts are removed 3 days after transplantation; then each heart is perfused with a noncirculating modified Tyrode's solution which contains 117.0 mM NaCl, 23.0 mM NaHCO₃, 4.6 mM KCl, 0.8 mM NaH₂PO₄, 1.0 mM MgCl₂, 2.0 mM CaCl₂, and 5.5 mM glucose, equilibrated at 37° C., and oxidized with a gas mixture containing 95% O₂ and 5% CO₂. In addition, the noncirculating modified Tyrode's solution used in the BP/ADSC/SIN group further contains 37 μM SIN-1, and the noncirculating modified Tyrode's solution used in the BP/ADSC/S3I group further contains 37 μM S31-201. The perfusion time is 30 minutes.

At the end of the experiment, the ROS expression level, p-STAT3 ratio, and IL-10 content in the left ventricle of the heart of each group are measured, with the measurement method being the same as that of Embodiment 1.

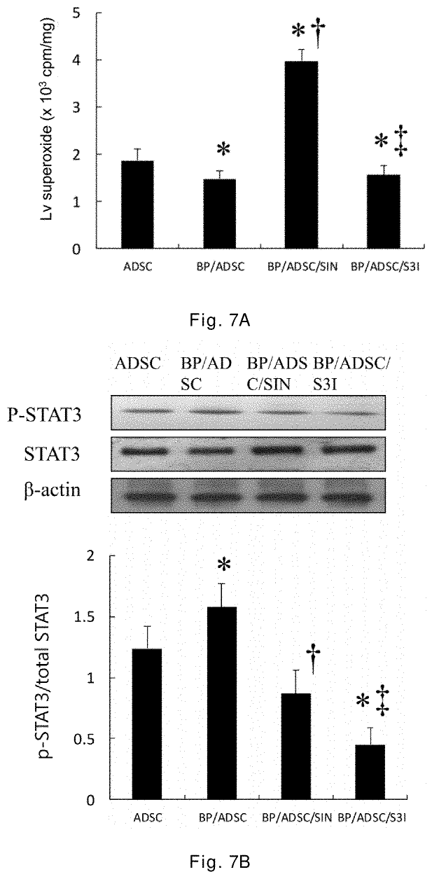

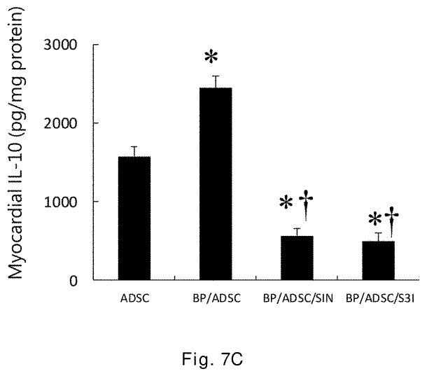

The results obtained are recorded in Table 10 and plotted as FIGS. 7A to 7C, respectively.

TABLE 10

| | ADSC | BP/ADSC | BP/ADSC/SIN | BP/ADSC/S3I |
|---|---|---|---|---|
| | group | group | group | group |
| number of rats | 5 | 5 | 5 | 5 |
| myocardial superoxide | 1.87 ± 0.24 | 1.48 ± 0.18 | 3.98 ± 0.25 | 1.56 ± 0.2 |
| (×10³ cpm/mg) | | | | |
| p-STAT3/total STAT3 | 1.24 ± 0.18 | 1.58 ± 0.19 | 0.87 ± 0.19 | 0.45 ± 0.14 |
| IL-10 (pg/mg protein) | 1576 ± 124 | 2453 ± 152 | 563 ± 98 | 493 ± 104 |

From the results of FIGS. 7A to 7C, it can be known that the p-STAT3/total STAT3 in the BP/ADSC/SIN group is 0.87±0.19 that decreases by about 45% as compared with the BP/ADSC group, and the IL-10 content of the BP/ADSC/SIN group is 563±98 μg/mg that decreases by about 77% as compared with the BP/ADSC group. Since SIN-1 is a peroxynitrite generator, these results show superoxide-mediated effects on the levels of p-STAT3 and IL-10.

Further, in order to evaluate whether the STAT3 signaling pathway is critical for the polarization of macrophage M2c, the effect of S3I-201 on the conversion of macrophages to M2c is analyzed. From the results of FIGS. 7A to 7C, it can be known that the IL-10 content of the BP/ADSC/S3I group is 493±104 μg/mg that decreases by about 80% (P<0.05) as compared with the BP/ADSC group Since S3I-201 is an inhibitor of STAT3, the results show the effect of STAT3 activation on the level of IL-10. Also, there is no significant difference when comparing the level of superoxide between the BP/ADSC group and the BP/ADSC/S3I group, indicating that the level of superoxide is not affected by the action of S3I-201, showing that the ROS is located upstream for regulating the STAT3 activation.

As described above, the content of the present invention has been specifically exemplified in the above-exemplified embodiments, but the present invention is not limited to the embodiments. Those having ordinary skills in the art to which the present invention pertains should understand that various changes and modifications can be made without departing from the spirit and scope of the present invention. For example, the technical contents exemplified in the foregoing embodiments are combined or changed to become a new embodiment, and these embodiments are of course considered as belonging to the present invention. Therefore, the scope of protection to be covered in this case also includes the scope of the claims described below and the scope defined by them.

What is claimed is:

1. A pharmaceutical composition for treating cardiac hypertrophy, comprising at least a stem cell and a pharmaceutically acceptable vehicle, wherein the stem cell is prepared by a pretreatment reaction of reacting with a n-butylidenephthalide (BP), and
the pharmaceutical composition is administered into a desired individual by remote intramuscular injection.

2. The pharmaceutical composition for treating cardiac hypertrophy according to claim 1, wherein the concentration of n-butylidenephthalide in the pretreatment reaction is in the range of 7 μg/mL to 40 μg/mL.

3. The pharmaceutical composition for treating cardiac hypertrophy according to claim 1, wherein the condition of the pretreatment reaction comprises reacting the stem cell with the n-butylidenephthalide at a temperature range of 20 to 40□ for 6 to 24 hours.

4. The pharmaceutical composition for treating cardiac hypertrophy according to claim 2, wherein the condition of the pretreatment reaction comprises reacting the stem cell with the n-butylidenephthalide at a temperature range of 20 to 40□ for 6 to 24 hours.

5. The pharmaceutical composition for treating cardiac hypertrophy according to claim 1, wherein the stem cell is at least one selected from a group consisting an umbilical cord blood stem cell, a peripheral blood stem cell, a neural stem cell, an adipose stem cell, and a bone marrow stem cell.

6. A method of using a stem cell in preparation of a pharmaceutical composition for treating cardiac hypertrophy, comprising administering an effective amount of a stem cell into a desired individual, wherein the stem cell is prepared by a pretreatment reaction of n-butylidenephthalide; and the desired individual is human or mammal.

7. The method of using a stem cell in preparation of a pharmaceutical composition for treating cardiac hypertrophy according to claim 6 wherein the pharmaceutical composition further comprises a pharmaceutically acceptable vehicle.

8. The method of using a stem cell in preparation of a pharmaceutical composition for treating cardiac hypertrophy according to claim 6, wherein an effective dose is in the range of 1×10⁶˜1×10⁸ stem cells.

9. The method of using a stem cell in preparation of a pharmaceutical composition for treating cardiac hypertrophy according to claim 6, wherein the cardiac hypertrophy is caused by spontaneous hypertension.

10. The method of using a stem cell in preparation of a pharmaceutical composition for treating cardiac hypertrophy according to claim 6, wherein the stem cell is administered via remote intramuscular injection.

11. The method of using a stem cell in preparation of a pharmaceutical composition for treating cardiac hypertrophy according to claim 10, wherein the distal muscle is a limb muscle or a buttock muscle.


SEQUENCE LISTING (16 sequences)

SEQ ID NO: 1 - DNA, 21 bp, Artificial Sequence
  human Alu sense primer
catggtgaaaccccgtctcta

SEQ ID NO: 2 - DNA, 19 bp, Artificial Sequence
  human Alu antisense primer
gcctcagcctcccgagtag

SEQ ID NO: 3 - DNA, 24 bp, Artificial Sequence
  IL-6 sense primer
ccagttgccttcttgggactgatg

SEQ ID NO: 4 - DNA, 24 bp, Artificial Sequence
  IL-6 antisense primer
attttctgaccacagtgaggaatg

SEQ ID NO: 5 - DNA, 25 bp, Artificial Sequence
  IL-1beta sense primer
atggcaactgtccctgaactcaact

SEQ ID NO: 6 - DNA, 26 bp, Artificial Sequence
  IL-1 beta antisense primer
caggacaggtatagattcaacccctt

SEQ ID NO: 7 - DNA, 20 bp, Artificial Sequence
  iNOS sense primer
tcaccttcgagggcagccga

SEQ ID NO: 8 - DNA, 20 bp, Artificial Sequence
  iNOS antisense primer
tccgtggcaaagcgagccag

SEQ ID NO: 9 - DNA, 21 bp, Artificial Sequence
  CD206 sense primer
tgggtttgctgaagaagagaa

SEQ ID NO: 10 - DNA, 24 bp, Artificial Sequence
  CD206 antisense primer
catgtgataagtgacaaatgcttg

SEQ ID NO: 11 - DNA, 21 bp, Artificial Sequence
  IL-10 sense primer
ggttgccaagccttgtcagaa

SEQ ID NO: 12 - DNA, 22 bp, Artificial Sequence
  IL-10 antisense primer
gctccactgccttgcttttatt

SEQ ID NO: 13 - DNA, 25 bp, Artificial Sequence
  cyclophilin sense primer
atggtcaaccccaccgtgttcttcg

SEQ ID NO: 14 - DNA, 25 bp, Artificial Sequence
  cyclophilin antisense primer
cgtgtgaagtcaccaccctgacaca

SEQ ID NO: 15 - DNA, 18 bp, Artificial Sequence
  BNP sense primer
agagagcaggacaccatc

SEQ ID NO: 16 - DNA, 19 bp, Artificial Sequence
  BNP antisense primer
aagcaggagcagaatcatc